(19) United States
(12) Patent Application Publication
(10) Pub. No.: US 2011/0173709 A1
(43) Pub. Date: Jul. 14, 2011

(54) ECTOPIC, ORTHOTOPIC MODEL FOR REVASCULARIZATION AND TUMOR ASSESSMENT

(75) Inventors: Jan E. Schnitzer, San Diego, CA (US); Philip Oh, San Diego, CA (US); Per Borgstrom, La Jolla, CA (US)

(73) Assignee: Sidney Kimmel Cancer Center

(21) Appl. No.: 12/529,339

(22) PCT Filed: Feb. 29, 2008
(86) PCT No.: PCT/US08/02727

§ 371 Date: Mar. 18, 2011

Related U.S. Application Data
(60) Provisional application No. 60/904,548, filed on Mar. 2, 2007.

(51) Int. Cl. A61K 49/00; A01K 67/00; A61K 31/713; A61K 31/7088; A61P 35/00; A61P 9/00

(52) U.S. Cl. 800/9; 800/8; 424/9.2; 514/44 A; 514/44 R

(57) ABSTRACT

Improved vascularization and tumor models, comprising a test animal having a dorsal skin window chamber, and an exogenous tissue sample implanted ectopically in the skin within the window chamber, are described, as are methods of using the models.

### RELATED APPLICATION

This application claims the benefit of U.S. Provisional Application No. 60/904,548, filed on Mar. 2, 2007. The entire teachings of the above application are incorporated herein by reference.

### GOVERNMENT SUPPORT

The invention was supported, in whole or in part, by grants RO1 HL52766, RO1HL58216, RO1HL074063, R24CA95893, P01CA104898 from the National Institutes of Health, and from grant 11RT-0167 from the Tobacco-Related Disease Research Program. The Government has certain rights in the invention.

### BACKGROUND OF THE INVENTION

Animal models are crucial to further our understanding of tumor biology. For many years, observation chambers implanted in various animal species have been used for intravital microscopy of tumor microcirculation. Transparent chambers have been instrumental in the understanding of tumor biology. With the development of molecular biology techniques such as spontaneously fluorescent proteins (GFP, m-Cherry), and elaborate image analysis software, it is now easier to generate quantitative data. Such systems can clarify tumor microcirculatory phenomena, and mechanisms underlying anti-angiogenic and anti-tumor activities that are poorly understood using traditional histopathology. Nevertheless, concerns remain regarding whether such animal models are accurate reflections of in vivo tumor growth and development.

### SUMMARY OF THE INVENTION

The present invention pertains to an improved vascularization model which comprises a test animal having a dorsal skin window chamber in which an exogenous tissue sample is implanted ectopically in the skin. The test animal can be, for example, a murine animal, and the exogenous tissue sample can be derived from an animal that is the same species as the test animal (e.g., from a body part of the same test animal), or can be derived from an animal that is a different species from the test animal (e.g., from a human individual). Representative exogenous tissue samples include, brain, breast, lung, kidney (renal), bladder, prostate, ovarian, head and neck, lymph, heart, and liver tissue samples.

The invention also pertains to an improved tumor model which comprises a test animal having a dorsal skin window chamber in which an exogenous tissue sample is implanted ectopically in the skin, and a tumor sample implanted in the exogenous tissue sample. The exogenous tissue sample and the tumor sample can be derived from the same type of tissue, or from different types of tissue.

The invention additionally pertains to methods for assessing an agent of interest for vascularization activity or for antitumor activity, in which the agent is administered to a vascularization model or to a tumor model. The agent of interest can be administered to the test animal, or directly to the exogenous tissue sample or tumor sample. Assessment of vascularization or of tumor characteristics after administration of the agent of interest, and comparison to vascularization or to tumor characteristics prior to administration of the agent of interest, indicates whether the agent of interest has vascularization activity or antitumor activity.

In addition, the invention pertains to methods for assessing a potential therapeutic target gene, by administering an agent that alters function of a gene of interest to a vascularization model or to a tumor model. Assessment of vascularization or of tumor characteristics after administration of the agent of interest, and comparison to vascularization or to tumor characteristics prior to administration of the agent of interest, indicates whether the gene of interest is therapeutic target gene. Representative agents that alter function of a gene of interest can include viral constructs (e.g., lentivirus constructs) comprising shRNA or siRNA for the gene of interest. The agent can be, for example, an agent that reduces protein expression of the gene of interest, or which increases protein expression of the gene of interest.

The invention further pertains to methods for increasing vascularization or for treating tumors in an individual by administering agents that alter function of a therapeutic target gene as identified by the methods herein.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A and 1B depict differential growth of N202 tumor spheroids in different engrafted tissue stromas. FIG. 1A: relative growth (intensity) over time.

FIG. 1B: relative growth (area) over time.

FIGS. 2A and 2B depict differential growth of LLC tumor spheroids in different engrafted tissue stromas. FIG. 2A: relative growth (intensity) over time. FIG. 2B: relative growth (area) over time.

FIG. 3 depicts vascular density of tumor progression. Fat pad, skin, lung and liver are compared.

FIG. 4 depicts a graphic representation of mitotic index versus apoptotic index.

FIGS. 5A, 5B and 5C depict vascular leakage due to tumor spheroid growth on different engrafted tissue stromas, as shown by leakage of Dextran (FIG. 5A) or by leakage of IgG (FIG. 5B). A comparison of the two is shown in FIG. 5C.

FIGS. 6A, 6B, 6C and 6D depict doxorubicin treatment of tumor spheroid growth on skin (FIG. 6A) and on fat pads (FIG. 6B), as well as for both skin and fat pads at concentrations of 1 mg/kg (FIG. 6C) and 5 mg/kg (FIG. 6D).

### DETAILED DESCRIPTION OF THE INVENTION

A description of example embodiments of the invention follows. The teachings of all patents, published applications and references cited herein are incorporated by reference in their entirety.

The present invention is drawn to an improved tumor model and an improved vascularization model, as well as to methods of assessing agents of interest and methods of assessing potential therapeutic target genes, in which the models and methods comprise a test animal having a dorsal skin window chamber with an exogenous tissue sample implanted ectopically in the skin within the window chamber.

The term, “test animal,” as used herein, refers to an animal, especially a mammal, that can be used for preparation of dorsal skin window chambers. In a particular embodiment, the test animal can be a mouse, such as a nude mouse. A wide variety of murine test animals can be used; other tests animals include rats, guinea pigs, and other appropriate animals. The test animal can be genetically modified, if desired (e.g., a transgenic or knockout animal).

A “tissue sample,” as used herein, is a set of cells which normally have a common function or occupy a common location in the body; for example, a tissue sample can be part of an organ. A tissue sample can also be from a tumor, and can include basic elements of a tissue such as stroma and cells (e.g., tumor cells). The term, “exogenous” as used herein (especially with reference to an “exogenous tissue sample”), refers to a tissue sample that is derived from a location other than the dorsal skin region of the test animal. The term, “derived from,” indicates that the tissue sample is taken from or obtained from a source (location), such as an organ, or is a sample that has been grown in cell culture from a tissue sample or from cells that have been taken from or obtained from a source (location) on an animal In certain embodiments, the tissue sample has been taken from or obtained from the test animal itself, and transplanted to the dorsal skin window chamber. In certain other embodiments, the tissue sample has been taken from or obtained from an animal other than the test animal. The animal from which the tissue sample derives can be the same species as the test animal, or can be a different species. For example, in one embodiment, the test animal is a nude mouse, and the tissue sample is a human tissue sample. In another embodiment, the test animal is a nude mouse, and the tissue sample is a rat tissue sample. In another embodiment, the test animal is a C57/black mouse, and the tissue sample is from a congenic mouse; alternatively, the test animal is a C57/black mouse, and the tissue sample is a tumor spheroid from LLC or B15 tumor cells. Any call type can be used. Representative exogenous tissue samples include, for example, brain, breast, lung, kidney (renal), bladder, prostate, ovarian, head and neck, lymph, heart, and liver tissue samples. The tissue sample can be from a genetically altered source, e.g., from a transgenic or knockout animal.

Certain embodiments of the invention relate to an improved model for vascularization comprising a test animal having a dorsal skin window chamber, in which an exogenous tissue sample is implanted ectopically in the skin within the window chamber. This model allows for enhanced study of vascularization in an exogenous tissue, which is useful for investigation of revascularization techniques (e.g., for transplant of organs or tissues). In addition, this improved model for vascularization can be used to assess agents of interest for their impact on the vascularization process itself, as well as to assess imaging agents useful for investigation of the vascularization process, as described below.

Another embodiment of the invention relates to an improved tumor model. The model comprises a test animal having a dorsal skin window chamber, in which an exogenous tissue sample is implanted ectopically in the skin within the window chamber; further, a tumor sample is implanted within the exogenous tissue sample. A tumor sample is a group of cells from a neoplasm. The term, “neoplasm,” as used herein refers particularly to malignant neoplasms, and includes not only to sarcomas (e.g., fibrosarcoma, myosarcoma, liposarcoma, chondrosarcoma, hemangiosarcoma, mesothelioma, leukemias, lymphomas, leiomyosarcoma, rhabdomyosarcoma), but also to carcinomas (e.g., adenocarcinoma, papillary carcinoma, cystadenocarcinoma, melanoma, renal cell carcinoma, hepatoma, choriocarcinoma, seminoma), as well as mixed neoplasms (e.g., teratomas). Thus, “neoplasm” contemplates not only solid tumors, but also so-called “soft” tumors. Furthermore, “neoplasm” contemplates not only primary neoplasms, but also metastases. In one preferred embodiment, the tumor sample is a tumor spheroid. The tumor tissue can be from the same species as the test animal and/or the exogenous tissue sample; alternatively, the tumor tissue can be from a different species from the test animal and/or the exogenous tissue sample. In representative embodiments, neoplasms that can be targeted include brain, breast, lung, kidney, bladder, prostate, ovarian, head and neck, and liver tumors. If desired, the tumor sample can comprise genetically modified tumor cells (e.g., expressing Histone2B-GFP, or alternatively or in addition, derived from a transgenic or knockout source). Tumor “characteristics” include, for example the size (e.g., volume), number, vascularization, encapsulation, and metastatic nature, of the tumor(s), and serve as indicia of the growth and development of the tumor.

This improved tumor model allows for enhanced study of tumor development in an exogenous tissue, which can be used to assess agents of interest for their impact on the tumor characteristics, as well as to assess imaging agents useful for investigation of the tumors, as described below.

Using either the vascularization model or the tumor model described above, the effect of an agent of interest can be assessed. An “agent of interest” is an agent to be tested for potential therapeutic activity. Representative agents include, for example, natural ligands, peptides, small molecules (e.g., inorganic small molecules, organic small molecules, derivatives of small molecules, composite small molecules); aptamers; cells, including modified cells; vaccine-induced or other immune cells; nanoparticles (e.g, lipid or non-lipid based formulations); lipids; lipoproteins; lipopeptides; lipid derivatives; liposomes; modified endogenous blood proteins used to carry chemotherapeutics; a protein (e.g., a recombinant protein or a recombinant modified protein) a carrier protein (e.g., albumin, modified albumin); a lytic agent; a small molecule; other nanoparticles (e.g., albumin-based nanoparticles, gold, dendrimers, carbon-based nanostructures); transferrins; immunoglobulins (antibodies); multivalent antibodies; analogues to antibodies (e.g., affibodies, minibodies); lipids; lipoproteins; liposomes; an altered natural ligand; a gene or nucleic acid; RNA, shRNA or siRNA; a viral or non-viral gene delivery vector; an antibody drug (e.g., avastin); a tyrosine kinase inhibitor, a prodrug; drug; or a promolecule. For example, the agent of interest can comprise a nucleic acid or ribonucleic acid construct that reduces protein expression of a gene (e.g., comprising siRNA or shRNA). Representative constructions can comprise lentiviral constructs; adenoviral constructs; adeno-associated virus (AAV) constructs; or other constructs.

In certain preferred embodiments, the agent of interest may alter function of a gene of interest and/or its encoded protein or peptide. A “gene of interest” is a gene which may be a potential therapeutic target gene; agents of interest that may alter the function of the gene of interest may change the transcription, translation, or protein expression resulting from that gene of interest. Alteration in function may result in increased transcription, translation, or expression; alternatively, it may result in decreased transcription, translation, or expression. A therapeutic target gene is a gene that is the target for altered expression, translation, or activity of the encoded protein, in order to achieve a particular effect. For example, in certain embodiments, the therapeutic target gene is a gene which affects growth and/or development of tumors, so that alteration of the expression of the gene or of the activity the encoded protein yields antitumor activity. In other embodiments, the therapeutic target gene is a gene which affects growth and/or development of vasculature, so that alteration of the expression of the gene or of the activity of the encoded protein yields activity which either enhances, or reduces, vascularization or re-vascularization. Target genes which affect growth and/or development of vasculature are useful not only for tumors, but also for normal (non-neoplastic) transplanted tissue or other normal tissue which undergoes revascularization.

Agents of interest can also include imaging agents. The imaging agent can comprise, for example, any of the agents described above. Alternatively or in addition, the imaging agent can comprise, for example, a radioactive agent (e.g., radioiodine (125I, 131I); technetium; yttrium; 35S or 3H) or other radioisotope or radiopharmaceutical; a contrast agent (e.g., gadolinium; manganese; barium sulfate; an iodinated or noniodinated agent; an ionic agent or nonionic agent); a magnetic agent or a paramagnetic agent (e.g., gadolinium, iron-oxide chelate); liposomes (e.g., carrying radioactive agents, contrast agents, or other imaging agents); nanoparticles; ultrasound agents (e.g., microbubble-releasing agents); a gene vector or virus inducing a detecting agent (e.g., including luciferase or other fluorescent polypeptide); an enzyme (horseradish peroxidase, alkaline phosphatase, ÿ-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); a bioluminescent material (e.g., luciferase, luciferin, aequorin); or any other imaging agent that can be employed for imaging studies (e.g., for CT, fluoroscopy, SPECT imaging, optical imaging, PET, MRI, gamma imaging).

In the methods of the invention, the agent of interest is administered to the vascularization model or to the tumor model. “Administration,” as used herein, can include, but is not limited to, intradermal, intramuscular, intraperitoneal, intraocular, intravenous, subcutaneous, topical, oral and intranasal, administration of the agent of interest to the test animal. Other suitable methods of introduction can also include rechargeable or biodegradable devices, particle acceleration devises (gene guns) and slow release polymeric devices. The agent can also be delivered directly to the exogenous tissue sample, or to the tumor sample, rather than (or in addition to) administration to the test animal itself. If desired, the agent can be administered after implanting the tissue sample, but prior to implanting the tumor sample, in the improved tumor model.

The agent can be administered by itself, or in a composition (e.g., a physiological or pharmaceutical composition) comprising the agent. For example, the agent can be formulated together with a physiologically acceptable carrier or excipient to prepare a pharmaceutical composition. The carrier and composition can be sterile. The formulation should suit the mode of administration. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents. The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc. If desired, the compositions can be administered into a specific tissue, or into a blood vessel serving a specific tissue (e.g., the carotid artery to target brain). The pharmaceutical compositions can also be administered as part of a combination with other agents, either concurrently or in proximity (e.g., separated by hours, days, weeks, months). Agents can also be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

After administration, the tissue sample in the vascularization model is assessed to determine whether the agent of interest has vascularization activity. “Vascularization activity,” as used herein, refers to increasing or enhancing angiogenesis or vascularization in the tissue sample. Vascularization activity can be identified by determining whether increased angiogenesis and/or vascularization occurs for the tissue sample.

In another embodiment, the tumor sample in the tumor model is assessed by examining the tumor characteristics, to determine whether the agent of interest has antitumor activity. The term, “antitumor activity” as used herein, can refer to reducing, preventing or delaying metastasis of the tumor; and/or reducing the number, volume, and/or size of one or more tumors; or otherwise causing a therapeutic change in the tumor characteristics. A “therapeutic change” indicates a change in the tumor characteristics that will decrease morbidity and mortality of the animal having the tumor (e.g., a change that is beneficial to survival of the animal having the tumor).

In either of these embodiments, the model can be assessed to determine whether the agent of interest has imaging activity. The term, “imaging activity” as used herein, can refer to physical imaging of an individual (e.g., the test animal) or of a part of the individual (e.g., the dorsal skin window chamber). Physical imaging, as used herein, refers to imaging of all or a part of an individual's body (e.g., by the imaging studies methods set forth above). Physical imaging can be positive, that is, can be used to detect the presence of a specific type of tissue or pathology (e.g., angiogenesis, neovasculature). For example, in one embodiment, positive physical imaging can be used to detect the presence or absence of a neoplasm, including the presence or absence of metastases, or to assess an individual for the presence or absence, or extent, or angiogenesis or of neovasculature. Alternatively, in another embodiment, positive physical imaging can be used to detect the presence or absence of a normal (non-disease) tissue, such as the presence of or absence of an organ. Alternatively, the physical imaging can be negative, that is, can be used to detect the absence of a specific type of tissue. For example, in one embodiment, negative physical imaging can be used to detect the absence or presence of a normal tissue, where the absence is indicative of a loss of function consistent with a pathology. Both positive and negative physical imaging permit visualization and/or detection of both normal and of abnormal pathology, and can be used to quantify or determine the extent, size, and/or number of an organ or of a type of neoplasm, as well as to quantify or determine the extent of angiogenesis or of neovasculature. Thus, an estimate can be made of the extent of disease or of angiogenesis or neovasculature, facilitating, for example, clinical diagnosis and/or prognosis.

In a further embodiment of the invention, the vascularization and tumor models can be used to assess a potential therapeutic target gene as described above. An alteration in vascularization activity or antitumor activity indicates that the gene of interest is a potential therapeutic target gene. In addition, the agents identifiable by the methods described herein can be used to increase vascularization or to treat tumors in individuals, by administration of the agents. In preferred embodiments, the agent is an agent that alters expression or activity of a gene of interest (e.g., an agent of interest, such as a lentiviral construct or other construct described herein).

### BENEFITS OF THE INVENTION

The models and methods of the invention allow investigation of agents that may alter (e.g., increase or decrease) the expression or function of a specific gene or protein or a set of genes or proteins, that then can be studied for their effects on revascularization or tumor development. For example, these tissue and tumor models can be used for genomic and proteomic analysis to assess key molecules being expressed at different times in revascularization or tumor development. Because the models more closely resemble natural in vivo conditions for vascularization and for tumors, data resulting from the methods and/or from genomic or proteomic analyses will be much more meaningful than when performed in currently available models, especially nonorthotopic subcutaneous tumor models.

The present invention is now illustrated by the following Exemplification, which is not intended to be limiting in any way. All references cited herein are incorporated by reference in their entirety.

### EXEMPLIFICATION

### Materials and Methods

Cell Lines—N₂₀₂ (Gift from Joseph Lustgarten, SKCC, San Diego) and LLC

(ATCC, Manassas, Va. 20108) cells were maintained in DMEM High Glucose supplemented with L-Glutamine (2 mM), Penicillin (100 U/ml), Streptomycin (100 U/ml), Sodium Pyruvate (1 mM) (Invitrogen, Carlsbad, Calif.) and 10% heat inactivated FBS (Omega Scientific, Tarzana, Calif.). TrampC2 (ATCC, Manassas, Va. 20108) were maintained as above except in RPMI1640 instead of DMEM High Glucose and supplemented as above with the addition of Insulin (5□g/ml) and dehydroisoandrosterone (10 nM) (Sigma, St. Louis, Mo.). Cultures were grown at 37° C. in 5% CO₂ in air.

Preparation of Tumor Spheroids—The above cells were transduced with a VSV pseudotyped LXRN virus encoding the Histone H2B fused to GFP. The histone H2B-GFP cDNA was subcloned into the SalI/HpaI sites in the LXRN vector (Clontech, Palo Alto, Calif.) using SalI and blunted NotI sites from the BOSH2BGFPN1 vector (Kanda et al 1998). The H2B-GFP containing virus was infected with VSV into GP-293 cells to produced viable virus containing the H2B-GFP. N₂₀₂, LLC and TrampC2 cells were transduced with the viable virus containing the H2B-GFP to stably incorporate the H2B-GFP gene. The transduced cells were FACs sorted 2× to ensure 100% of the cells stably expressed the H2B-GFP protein. Tumor spheroids were formed by the addition of 50,000 cells onto 1% agar coated 96 well non tissue culture treated flat bottom dishes. Cells were forced together to form the spheroid by centrifugation at 2000 rpm for 15 minutes (4×) rotating the dish after every centrifugation. The cells were allowed to form the tumor spheroid for 2-5 days (depending on cell type) prior to implantation into the dorsal skinfold window chamber.

Dorsal Skinfold Window Chamber—All animals experiments were performed in accordance with Sidney Kimmel Cancer Center IACUC guidelines. Athymic and T-cell deficient nu/nu nude mice (both male—TrampC2 and LLC—and female—N202 and LLC) from Charles River Laboratories (Wilmington, Mass.) were used in our studies. The dorsal skinfold window chamber was prepared as previously described (Frost, G. I., et al. Novel syngeneic pseudo-orthotopic prostate cancer model: vascular, mitotic and apoptotic responses to castration. Microvasc Res 69, 1-9 (2005)). In short, the mice (25-30 g body weight) were anesthetized (7.3 mg ketamine hydrochloride and 2.3 mg xylazine per 100 g body weight, intraperitoneal injection) and placed on a heating pad. Two symmetrical titanium frames were placed onto the dorsal skinfold of the mice so as to sandwich the extended double layer of skin. A 15 mm diameter full-thickness circular layer was then excised. The underlying muscle and subcutaneous tissues were covered with a glass coverslip incorporated to one of the two frames. After a recovery period of 1-3 days, tumor spheroids were implanted into the dorsal skinfold window chamber.

Tumor Spheroid Implantation—Mammary fat pad from a lactating female mouse, lung (either male or female), liver (either male or female) and prostate tissue from a male mouse was excised and minced into small pieces. The excised minced tissues, one type per chamber, were implanted in the dorsal skinfold chambers. The tumor spheroids were placed upon the engrafted tissue stroma. In the case of the skin, the tumor spheroids were placed directly onto the skin of the dorsal skinfold chamber.

### Results

Tumor progression and revascularization—The goal was to determine the importance of the tissue stroma for tumor progression. N202, murine mammary adenocarcinoma cells, and Lewis Lung Carcinoma (LLC), murine lung carcinoma cells, and TrampC2, murine prostate adenocarcinoma cells, were transduced with a VSV pseudo-typed LXRN virus encoding the histone H2B-GFP fusion protein (Frost, G. I., et al. Novel syngeneic pseudo-orthotopic prostate cancer model: vascular, mitotic and apoptotic responses to castration. Microvasc Res 69, 1-9 (2005)). This allowed us to follow tumor progression by the analysis of the growth of the tumor as well as the intensity of the GFP signal due to cell division.

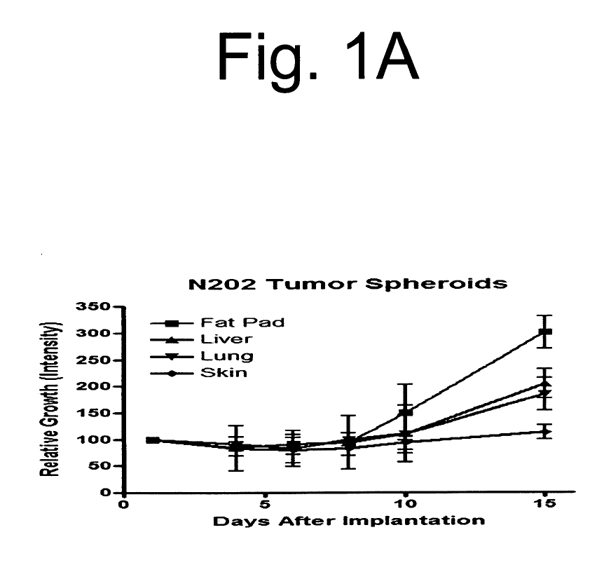

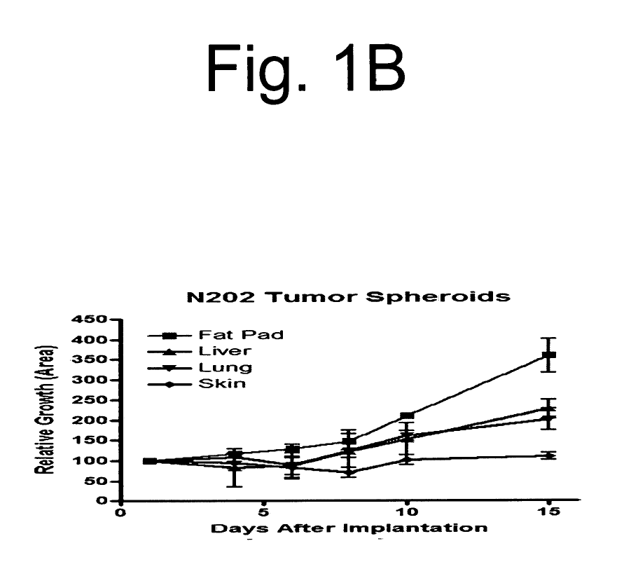

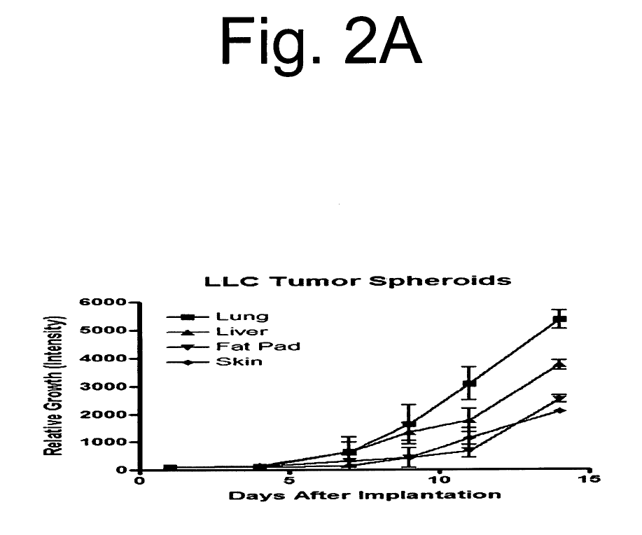

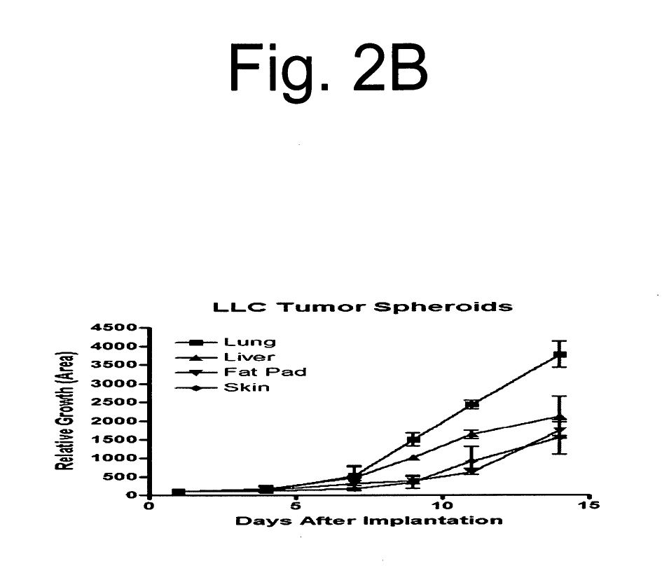

FIGS. 1A-B and 2A-B show graphic representations of tumor progression over time for N202 (FIGS. 1A and 1B) and LLC (FIGS. 2A and 2B). Tumor progression is shown either by the intensity of the GFP signal of at least 3 animals per tumor tested (FIG. 1A, 2A) or by the area of the tumor as outlined by the GFP signal (FIG. 1B, 2B). To follow the tumor progression, tumor spheroids consisting of the indicated cells were implanted either subcutaneously on the dorsal skinfold or on the indicated engrafted tissues. In all cells tested, when the tumor spheroid was implanted on its engrafted orthotopic stroma, the mammary fat pad from a lactating female donor mouse for the N202, the lung for the LLC and the prostate for the TrampC2, as compared with the skin alone or even engrafted non-orthotopic stromas, lung, liver and fat pad, there appeared to be more rapid tumor growth and progression as indicated by both area and intensity of the GFP signal in all studied cases. N202 H2B-Cherry was implanted on the engrafted mammary fat pad tissue stroma from a lactating GFP mouse. Tumor progression was studied over time to determine the derivation of the tumor vasculature. Results (not shown) demonstrated that the tumor vasculature is derived from the engrafted stroma, as indicated by the GFP labeling of the growing tumor vessels, further indicating the importance of the engrafted tissue stroma.

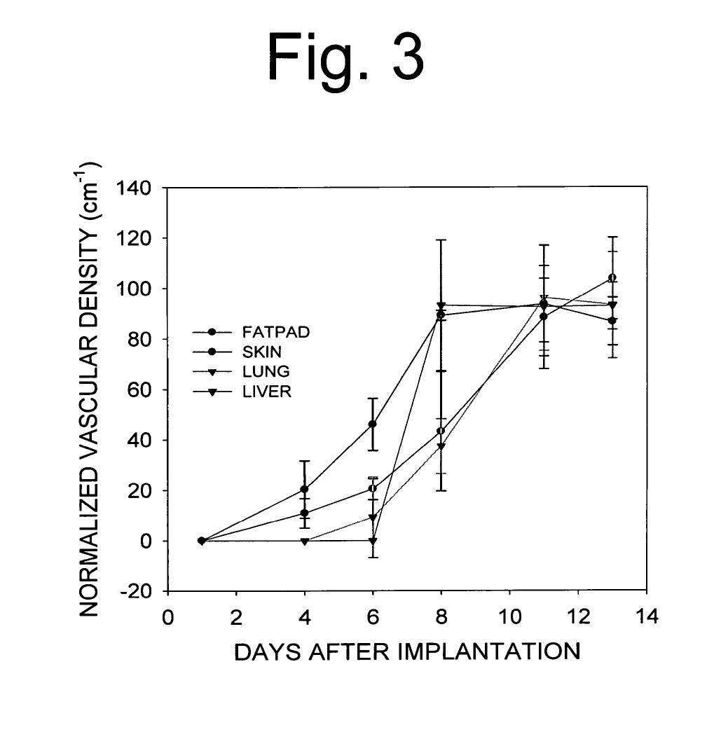

Vascular Density of tumor progression—N202, murine mammary adenocarcinoma cells containing the H2B-GFP fusion protein were implanted either subcutaneously on the dorsal skinfold or on the indicated engrafted tissues. We followed the re-vascularlization of the progressing tumors and determined the vascular density. FIG. 3 graphically illustrates the normalized vascular density of the N202 tumors in the indicated engrafted tissues. When the N202 tumors are implanted in their orthotopic stroma, the mammary fat pad, re-vascularization occur earlier reaching what appears to be complete re-vascularization more rapidly than the other engrafted tissues. Interesting, the N202 tumors implanted in the engrafted liver initially showed little to no re-vascularization followed by rapid re-vascularization to what appears to be complete re-vascularization with 2-3 days but the occurrence of the re-vascularization was later than what was seen when the N202 are grown in the mammary fat pad. This re-vascularization dependency was also seen in the other two tumor cell lines studied with similar results of the engrafted orthotopic stroma have the more rapid re-vascularization as compared with either the skin or the non-orthotopic stromas (data not shown).

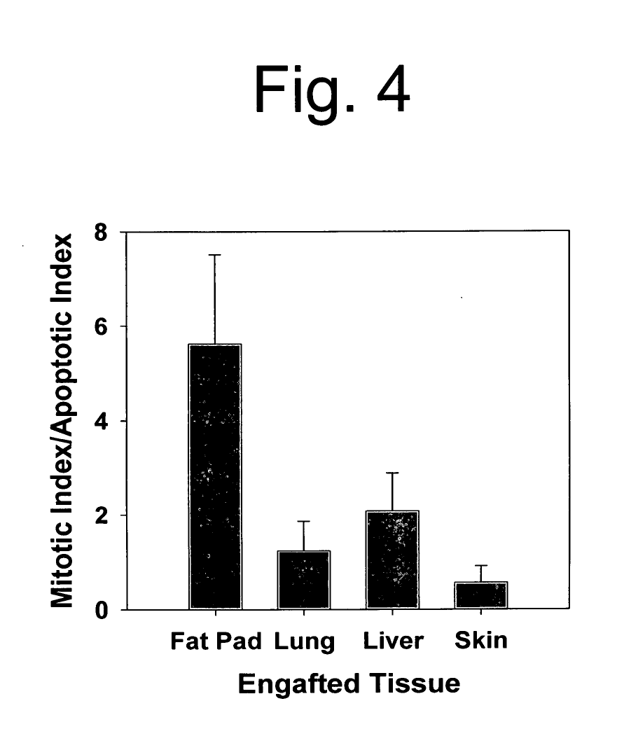

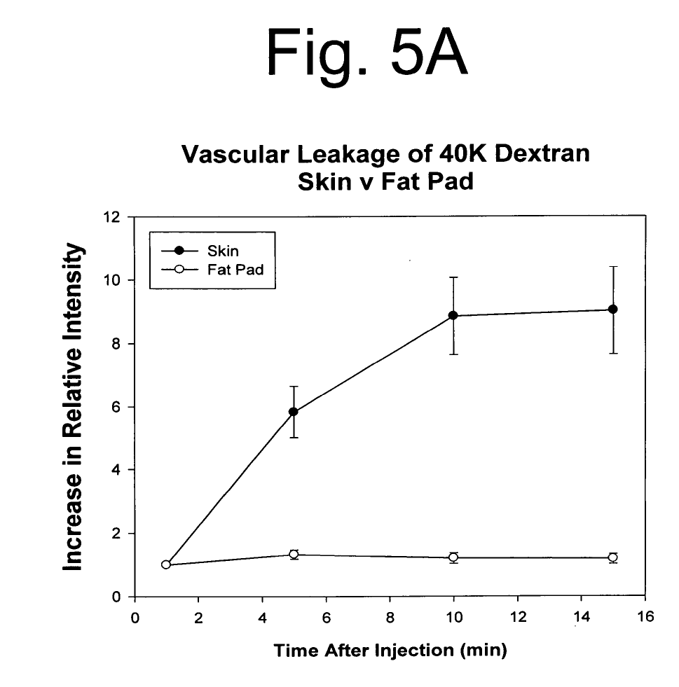

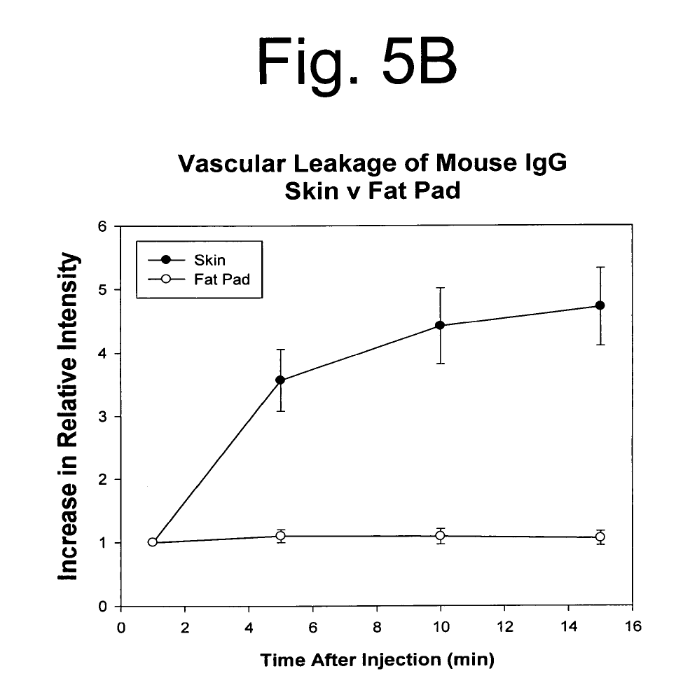

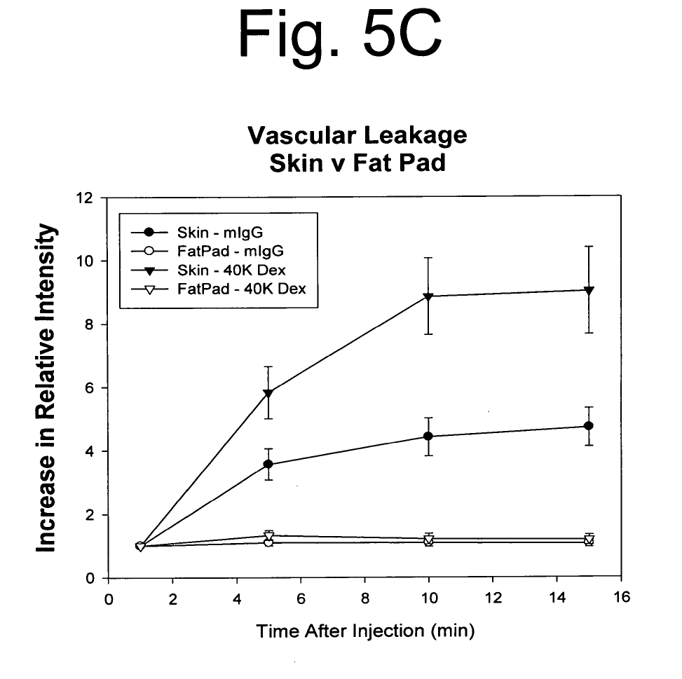

Orthotopic versus non-orthotopic implantation—The progressing tumors were observed at higher magnifications to see if there were any visual differences between orthotopic versus non-orthotopic implantation. At higher magnification, there seemed to be more penetration of tumor cells into the orthotopic stroma, as seen with an organized migration of the tumor cells into the orthotopic stroma, while in the case of the implanted tumor on the skin there seemed to be more encapsulation of the progressing tumor (not shown). In the case of the implantation on non-orthotopic stroma, there was tumor cell migration, but it seemed to be more disorganized. This was also seen when one implanted the tumor spheroid on the edge of the orthotopic stroma. The tumor cells migrated only towards the orthotopic stroma while being encapsulated on the opposite edge (data not shown). Because the GFP was fused to the histone H2B protein, mitosis as well as apoptosis of the tumor cells could be followed. Higher magnifications showed the effect of the stroma on the LLC-H2BGFP, as the progressing tumor grown in its orthotopic stroma seemed to be more polarized with cells undergoing mitosis while when the tumor spheroid was grown in the other stomas, the cells appeared more round with apoptotic cells. Similar observations were seen in all cases for the tumor spheroids studied (data not shown). Therefore, even though the tumor spheroid had the ability to grow in the non-orthotopic stroma, there was preferential growth in the orthotopic stroma as seen with both the organized migration of the tumor cells into the orthotopic stroma as well as greater mitosis. FIG. 4 depicts a graphic representation of mitotic index versus apoptotic index. There appeared to be a correlation between the progression of the tumor and the ratio of mitotic index versus apoptotic index with the greater ratio having more rapid tumor progression both re-vascularization and growth. Leaky tumor vasculature—It was decided to assess whether this animal model had “tumor vascular permeability”. As depicted in FIG. 5A, when we implanted the N202-H2BGFP tumor spheroids directly on the skinfold, we were able to observe 40 kD Dextran moving from the tumor vasculature into the underlying tumor. But when we implanted the tumor spheroids on engrafted orthotopic tissue stroma, we observed little to no Dextran in the underlying tumor with the Dextran signal remaining within the vasculature. We decided to see if we could observe similar results as the Dextran with mouse IgG (mIgG) to determine if there was a size determinant to the observed “tumor vascular permeability”. We observed the mouse IgG moving into the underlying tissue in the tumor spheroids implanted directly on the skinfold with little to no observable mIgG signal in the underlying tumor when implanted on the engrafted orthotopic tissue stroma (FIG. 5B). FIG. 5C is a graphic representation of the comparison of movement of Dextran and of IgG. Therefore we determined that the observed event wasn't “tumor vascular permeability” but that the skinfold implantation was most likely just leakier than orthotopic stroma implantation.

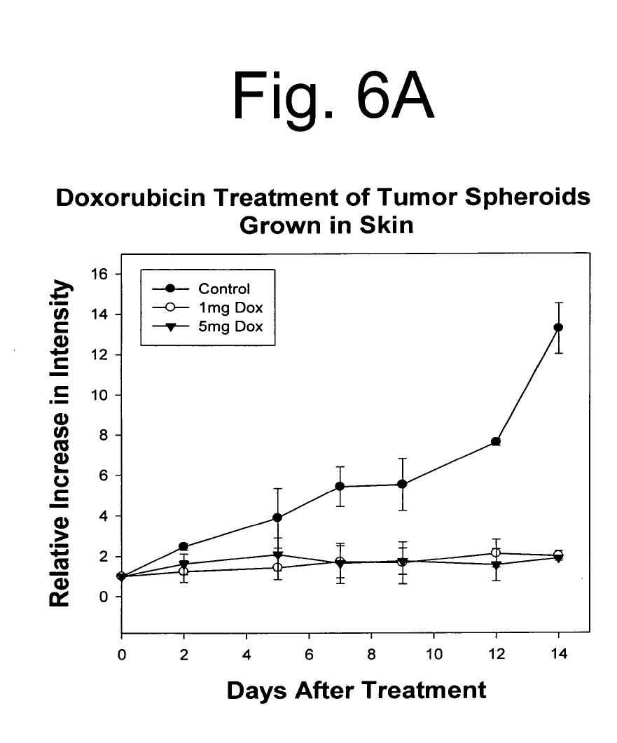

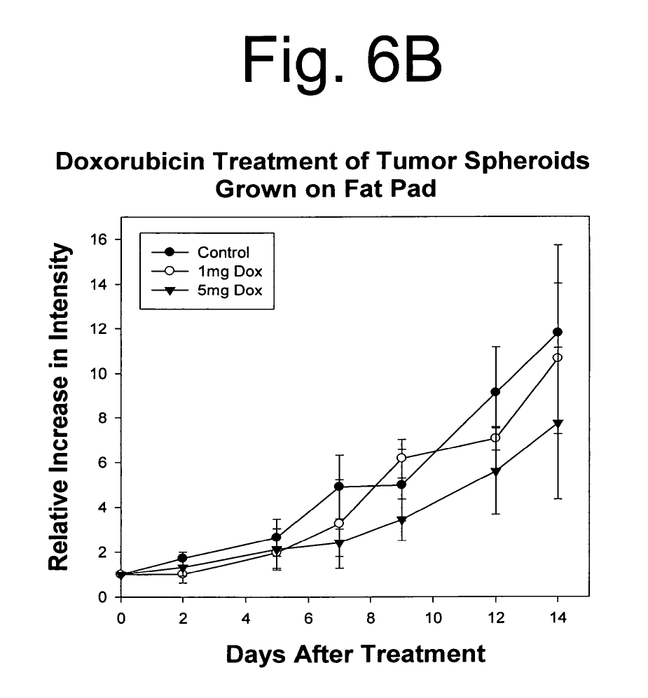

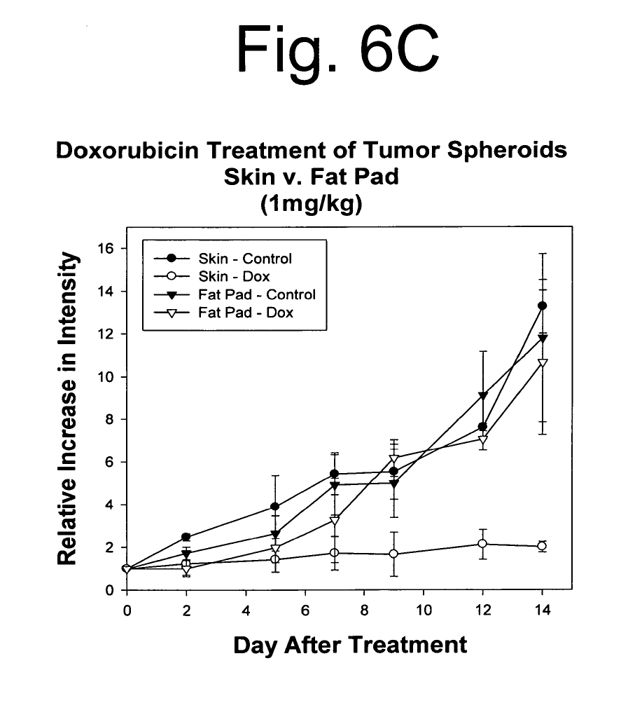

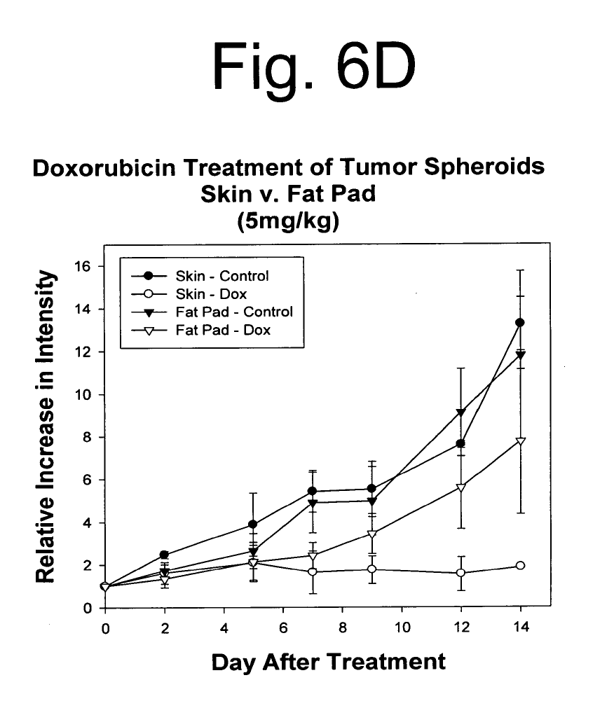

Bias of drug efficacy—As described above, the tumor vasculature of the growing tumor implanted on its orthothopic stroma was less leaky than the tumor vasculature of the growing tumor implanted on the skinfold. We decided to investigate if this apparent leakiness may be one of causes of the propensity for false-positive pre-clinical results in subcutaneous animal models. We implanted N202-H2BGFP tumor spheroids in either engrafted mammary fat pad or the skinfold itself. After allowing the growing tumor to re-vascularize, we added a single dose of Doxorubicin at 1 or 5 mg/kg via the tail vein. Similar to many pre-clinical studies in subcutaneous animal models, this single dose had a pronounced effect on the growing tumor implanted on the skinfold (FIG. 6A), having essentially tumor stasis, while having seemingly little to no effect on the growing tumor implanted on the engrafted mammary fat pad over a 2 week period (FIG. 6B). The comparison of 1 mg/kg for both fat pad and skin is shown in FIG. 6C; the comparison of 5 mg/kg is shown in FIG. 6D. These results were similar to what has been observed both pre-clinically (skin implantation) and the clinic (orthotopic implantation).

While this invention has been particularly shown and described with references to example embodiments thereof, it will be understood by those skilled in the art that various changes in form and details may be made therein without departing from the scope of the invention encompassed by the appended claims.

What is claimed is:

1. An improved vascularization model, comprising a dorsal skin window chamber of a test animal, wherein an exogenous tissue sample is implanted ectopically in the skin within the window chamber.

2. The model of claim 1, wherein the test animal is murine.

3. The model of claim 1, wherein the exogenous tissue sample is derived from an animal that is the same species as the test animal.

4. The model of claim 3, wherein the exogenous tissue sample is transplanted from a body part of the same test animal.

5. The model of claim 1, wherein the exogenous tissue sample is derived from an animal that is a different species from the test animal.

6. The model of claim 5, wherein the exogenous tissue sample is from a human individual.

7. The model of claim 1, wherein the exogenous tissue sample is selected from the group consisting of: example, brain, breast, lung, kidney (renal), bladder, prostate, ovarian, head and neck, lymph, heart, and liver tissue samples.

8. A method for assessing an agent of interest for vascularization activity, comprising:
a) administering the agent to a test animal having a dorsal skin window chamber, wherein an exogenous tissue sample is implanted within the window chamber;
b) assessing the vascularization of the exogenous tissue sample after administering the agent; and
c) comparing the vascularization to vascularization of the exogenous tissue sample prior to administering the agent,
wherein an alteration in the vascularization is indicative that the agent of interest has vascularization activity.

9. The method of claim 8, wherein the agent is administered directly to the exogenous tissue sample.

10. The method of claim 8, wherein the agent is administered to the test animal.

11. A method for assessing a potential therapeutic target gene, comprising:
a) administering an agent that alters function of a gene of interest to a test animal having a dorsal skin window chamber, wherein an exogenous tissue sample is implanted within the window chamber;
b) assessing the vascularization of the exogenous tissue sample after administering the agent; and
c) comparing the vascularization to vascularization of the exogenous tissue sample prior to administering the agent,
wherein an alteration in the vascularization is indicative that the gene of interest is therapeutic target gene.

12. The method of claim 11, wherein the agent that alters function of the gene of interest comprises a viral construct comprising shRNA or siRNA for the gene of interest.

13. The method of claim 12, wherein the viral construct comprises a lentivirus construct.

14. The method of claim 11, wherein the agent that alters function of the gene of interest comprises a nucleic acid construct that reduces protein expression of the gene of interest.

15. The method of claim 11, wherein the agent that alters function of the gene of interest comprises a nucleic acid construct that increases protein expression of the gene of interest

16. An improved tumor model, comprising a dorsal skin window chamber of a test animal, wherein an exogenous tissue sample is implanted ectopically in the skin within the window chamber, and a tumor sample is implanted within the exogenous tissue sample.

17. The model of claim 16, wherein the test animal is murine.

18. The model of claim 16, wherein the exogenous tissue sample is derived from an animal that is the same species as the test animal.

19. The model of claim 18, wherein the exogenous tissue sample is transplanted from a body part of the same test animal.

20. The model of claim 16, wherein the exogenous tissue sample is derived from an animal that is a different species from the test animal.

21. The model of claim 20, wherein the exogenous tissue sample is from a human individual.

22. The model of claim 16, wherein the exogenous tissue sample is selected from the group consisting of: example, brain, breast, lung, kidney (renal), bladder, prostate, ovarian, head and neck, lymph, heart, and liver tissue samples.

23. The model of claim 1, wherein the exogenous tissue sample and the tumor sample are derived from the same type of tissue.

24. The model of claim 1, wherein the exogenous tissue sample and the tumor sample are derived from the different types of tissue.

25. A method for assessing an agent of interest for antitumor activity, comprising:
a) administering the agent to a test animal having a dorsal skin window chamber, wherein an exogenous tissue sample is implanted within the window chamber, and a tumor sample is implanted within the exogenous tissue sample;
b) assessing the tumor characteristics after administering the agent; and
c) comparing the tumor characteristics after administering the agent to the tumor characteristics prior to administering the agent,
wherein the presence of a therapeutic change in the tumor characteristics is indicative of antitumor agent by the agent of interest.

26. The method of claim 25, wherein the agent is administered directly to the exogenous tissue sample.

27. The method of claim 25, wherein the agent is administered directly to the tumor sample.

28. The method of claim 25, wherein the agent is administered to the test animal.

29. A method for assessing a potential therapeutic target gene, comprising:
a) administering an agent that alters function of a gene of interest to a test animal having a dorsal skin window chamber, wherein an exogenous tissue sample is implanted within the window chamber, and a tumor sample is implanted within the exogenous tissue sample;
b) assessing tumor sample characteristics after administering the agent; and
c) comparing tumor sample characteristics after administering the agent to the tumor sample characteristics prior to administering the agent,
wherein the presence of a therapeutic change in the tumor characteristics is indicative that the gene of interest is therapeutic target gene.

30. The method of claim 29, wherein the agent that alters function of the gene of interest comprises a viral construct comprising shRNA or siRNA for the gene of interest.

31. The method of claim 30, wherein the viral construct comprises a lentivirus construct.

32. The method of claim 29, wherein the agent that alters function of the gene of interest comprises a nucleic acid construct that reduces protein expression of the gene of interest.

33. The method of claim 29, wherein the agent that alters function of the gene of interest comprises a nucleic acid construct that increases expression of the gene of interest.

34. A method for treating a tumor in an individual, comprising administering an agent identifiable by the method of claim 25 to the individual.

35. The method of claim 34, wherein the agent comprises a viral construct comprising shRNA or siRNA for a gene of interest.

36. The method of claim 35, wherein the viral construct comprises a lentivirus construct.

37. The method of claim 34, wherein the agent comprises a nucleic acid construct that reduces expression of a gene of interest.

38. The method of claim 34, wherein the agent comprises a nucleic acid construct that increases expression of a gene of interest.

39. A method for increasing vascularization in an individual, comprising administering an agent identifiable by the method of claim 8 to the individual.

40. The method of claim 39, wherein the agent comprises a viral construct comprising shRNA or siRNA for a gene of interest.

41. The method of claim 40, wherein the viral construct comprises a lentivirus construct.

42. The method of claim 39, wherein the agent comprises a nucleic acid construct that reduces expression of a gene of interest.

43. The method of claim 39, wherein the agent comprises a nucleic acid construct that increases expression of a gene of interest.
